(19) United States
(12) Patent Application Publication
(10) Pub. No.: US 2026/0256668 A1
(43) Pub. Date: Sep. 3, 2026

(54) METHODS FOR STRENGTHENING AND REPAIRING HAIR

(71) Applicant: Living Proof, Inc., Boston, MA (US)

(72) Inventors: Zhaoxia Ji, Natick, MA (US); Nawodi Abeyrathna, Boston, MA (US); Ronald P. McLaughlin, Boston, MA (US)

(21) Appl. No.: 19/158,121

(22) PCT Filed: Feb. 21, 2024
(86) PCT No.: PCT/US2024/016648

§ 371 Date: Aug. 20, 2025

Related U.S. Application Data
(60) Provisional application No. 63/447,404, filed on Feb. 22, 2023.

(51) Int. Cl. A61K 8/55; A61K 8/24; A61Q 5/00

(52) U.S. Cl. CPC A61K 8/55; A61K 8/24; A61Q 5/002

(57) ABSTRACT

Provided are compositions comprising ionizable phosphorus containing compounds and their use to strengthen and/or repair hair.

### RELATED APPLICATIONS

This application claims priority to U.S. Provisional Application No. 63/447,404 filed Feb. 22, 2023, the entire contents of which are incorporated herein by reference.

### BACKGROUND

General hair practices of consumers, such as regular washing cycles, mechanical and chemical processing, thermal styling, and other environmental factors can lead to various types of hair damage, both on the hair surface and inside the cortex. To address these problems and repair hair damage, it is important to develop effective technologies that not only provide hair surface repair but also internal structural strengthening.

### SUMMARY

Treating damaged hair with certain compounds having one or more phosphorus containing molecules has now been found to result in significant improvements in the thermal and/or mechanical properties of the hair. See e.g., FIGS. 1-4, 6-9, 15, and 17, where treatment of hair with a series of ionizable phosphorus containing molecules was found to improve the denaturation temperature (Td), Young's modulus, and break stress.

Additionally, it was also found that certain compounds having one or more ionizable phosphorus containing groups improved surface hydrophobicity. See e.g., FIGS. 5, 10, 12, 14, 16, and 18.

Further results and data are provided in the Exemplification section below.

In one aspect, therefore, provided herein are hair care compositions comprising phosphorus containing compounds such as ionizable phosphorus containing compounds (e.g., hydrocarbon based phosphonic acids, hydrocarbon-based phosphates, complex lipids, hydrocarbon-based phosphate esters, or polyphosphates, or combinations thereof), as well as their use for strengthening or repairing hair (e.g., the hair on the head of human).

### BRIEF DESCRIPTION OF THE DRAWINGS

FIG. 1 shows the hair denaturation temperature of 3× bleached hair treated with a series of ionizable phosphorus containing molecules with multi-phosphonic acid groups at 2 wt %, pH 2 compared to virgin, and bleached hair.

FIG. 2 shows the hair denaturation temperature of 3× bleached hair treated with Diethylenetriamine penta-(methylene phosphonic acid) (DTPMP) at various concentrations compared to phytic acid treated, virgin, and bleached hair.

FIG. 3 shows the percent change in hair mechanical properties of 3× bleached hair treated with DTPMP solutions at different active concentrations compared to bleached hair before the treatment.

FIG. 4 shows the hair denaturation temperature of 3× bleached hair treated with a series of ionizable phosphorus containing molecules with multi-phosphonic acid groups at 2 wt %, pH 4-4.5 compared to virgin and bleached hair.

FIG. 5 shows the water contact angle of 1× bleached hair treated with ionizable phosphorus containing molecules with multi-phosphonic acid groups at 2 wt % concentration, compared to untreated virgin, and bleached hair.

FIG. 6 shows the hair denaturation temperature of 3× bleached hair treated with a series of ionizable phosphorus containing molecules with at least one other functional group at 2 wt %, pH 4-4.5 compared to phytic acid treated, virgin, and bleached hair.

FIG. 7 shows the hair denaturation temperature of 3× bleached hair treated with a series of linear and cyclic polyphosphates at 2 wt %, pH 4-4.5 compared to phytic acid treated, virgin, and bleached hair.

FIG. 8 shows the percent change in hair mechanical properties of 3× bleached hair treated with DTPMP, AMPA, VPA, OPA and TSPP compared to the bleached hair before treatment.

FIG. 9 shows the hair denaturation temperature of 3× bleached hair treated with a series of alkyl phosphonic acids at 2 wt %, compared to phytic acid treated, virgin, and bleached hair.

FIG. 10 shows the water contact angle of 1× bleached hair treated with a series of alkyl phosphonic acids at 2 wt %, compared to untreated virgin and bleached hair.

FIG. 11 shows the Coefficient of Friction (COF) of 18-MEA removed hair treated with a series of alkyl phosphonic acids at 2 wt %, compared to untreated Virgin and bleached hair.

FIG. 12 shows the water contact angle of 3× bleached hair treated with 2 wt % lauryl phosphate (DPhate) or stearyl phosphate (ODPhate) as compared to untreated virgin and 3× bleached hair.

FIG. 13 shows the coefficient of friction (COF) of 18-MEA removed hair treated with DPhate or ODPhate at 2 wt %, compared to untreated virgin and 18-MEA removed hair.

FIG. 14 shows the water contact angle of 3× bleached hair treated with a series of complex lipids at 1 wt %, compared to untreated virgin and bleached hair.

FIG. 15 shows the hair denaturation temperature of 3× bleached hair treated with mixtures of DPA and VPA, compared to 2 wt % DPA alone, untreated virgin and bleached hair.

FIG. 16 shows the water contact angle of 1× bleached hair treated with mixtures of DPA and VPA, compared to 2 wt % VPA alone, untreated virgin and bleached hair.

FIG. 17 shows the hair denaturation temperature of 3× bleached hair treated with base formula containing a mixture of 0.75 wt % PA and 1 wt % Lauryl phosphate (DPhate), compared to base formula, base formula with 0.75 wt % PA alone, untreated virgin and bleached hair.

FIG. 18 shows the water contact angle of 3× bleached hair treated with base formulas containing a mixture of 0.75% PA with 1 wt % Lauryl phosphate (DPhate), compared to base formula, base formula with 0.75 wt % PA alone, untreated virgin and bleached hair.

FIG. 19 shows the coefficient of friction (COF) of 18-MEA removed hair treated with base formulas containing a mixture of 0.75 wt % PA and 1 wt % Lauryl phosphate (DPhate), compared to base formula, base formula with PA alone, untreated virgin and 18-MEA removed hair.

### DETAILED DESCRIPTION

In one aspect, provided are hair care compositions comprising an effective amount of an ionizable phosphorus containing compound selected from a hydrocarbon based phosphonic acid, a hydrocarbon based phosphate, a hydrocarbon based phosphate ester, a complex lipid, or a polyphosphate, or a combination of any of the foregoing.

In one aspect, also provided are methods for strengthening and/or repairing human hair comprising applying to the hair a hair care composition described herein. In one aspect, the described compositions are applied to hair that has been damaged. In another aspect, the described compositions are applied to hair that has been bleached. In yet another aspect, the described compositions are applied to hair that has been reduced (e.g., with a reducing agent such as ammonium thioglycolate). In yet another aspect, the described compositions are applied to hair that has been color treated. In yet another aspect, the described compositions are applied to hair that has been heat treated.

As used herein, the term ionizable phosphorus containing compound refers to a compound which comprises one or more ionizable phosphate groups, e.g., a —POH or —P(OH)₂ group. It will be appreciated that depending upon the pH of the medium, the ionizable phosphate group may be fully ionized, non-ionized, or partially ionized, each of which are encompassed by the term “ionizable.”

Phosphonic acid functional groups contain at least one phosphorous atom bonded to three oxygen atoms (two hydroxy moieties (P—OH) and one P═O double bond) and one carbon atom (P—C bond). Hydrocarbon based phosphonic acids refer to cyclic (aromatic or non-aromatic) or acyclic linear or branched chain compounds composed of a backbone having primarily hydrocarbon based units and one or more phosphonic acid groups, i.e.,

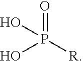

As further described herein, one or more hydrocarbon units of the backbone can be substituted with one or more suitable substituents and/or interrupted by one or more heteroatoms (e.g., N and O). In one embodiment, a hydrocarbon based phosphonic acid is acrylic. In one embodiment, a hydrocarbon based phosphonic acid comprises at least two phosphonic acid groups, at least three phosphonic acid groups, at least four phosphonic acid groups, or at least five phosphonic acid groups. In another embodiment, a hydrocarbon based phosphonic acid comprises from two to five phosphonic acid groups. In another embodiment, a hydrocarbon based phosphonic acid comprises two, three, four, or five phosphonic acid groups. In one embodiment, a hydrocarbon based phosphonic acid comprises one or more nitrogen atoms (optionally in addition to the one or more phosphonic acid groups described above). In another embodiment, a hydrocarbon based phosphonic acid comprises one, two, or three nitrogen atoms (optionally in addition to the one or more phosphonic acid groups described above). In one embodiment, a hydrocarbon based phosphonic acid comprises a nitrogen atom interrupting a hydrocarbon chain (optionally in addition to the one or more phosphonic acid groups or nitrogen atoms described above). In one embodiment, a hydrocarbon based phosphonic acid comprises an NH₂ group (optionally in addition to the one or more phosphonic acid groups and/or nitrogen atoms and/or nitrogen atom interrupting a hydrocarbon chain described above). In one embodiment, a hydrocarbon based phosphonic acid comprises an OH group optionally in addition to the one or more phosphonic acid groups and/or nitrogen atoms and/or nitrogen atom interrupting a hydrocarbon chain and/or NH₂ group described above). In one embodiment, a hydrocarbon based phosphonic acid comprises an COOH group optionally in addition to the one or more phosphonic acid groups and/or nitrogen atoms and/or nitrogen atom interrupting a hydrocarbon chain and/or NH₂ and/or OH group described above).

In one embodiment, a hydrocarbon based phosphonic acid is of the structural formula I, II, III, or IV:

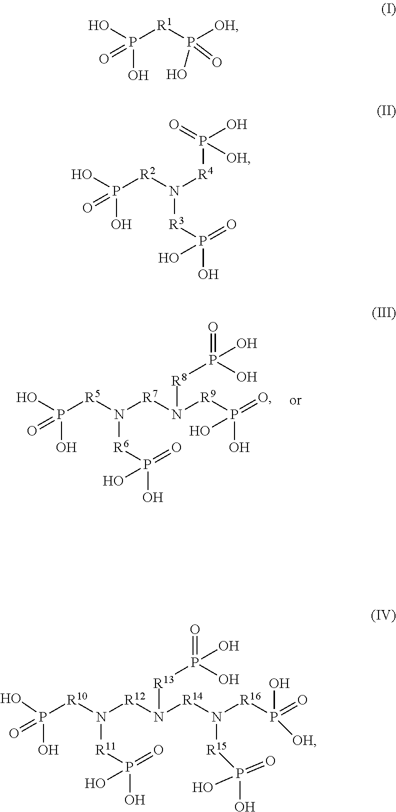

wherein each of R¹ to R⁸ are independently selected from an optionally substituted (C₁-C₆) alkylene. In one embodiment, each of R¹ to R¹⁶ in any one of Formula I, II, III, or IV are independently selected from an optionally substituted (C₁-C₃)alkylene. Alternatively, R¹ to R¹⁶ in any one of Formula I, II, III, or IV are independently selected from a (C₁-C₃)alkylene optionally substituted with OH. In another alternative, R¹ to R¹⁶ in any one of Formula I, II, III, or IV is a (C₁-C₃)alkylene optionally substituted with OH. In one embodiment, R¹ in any one of Formula I, II, III, or IV is a (C₁-C₃)alkylene optionally substituted with OH, wherein the remaining variables are as described in any of the foregoing embodiments or alternative. In one embodiment, a hydrocarbon based phosphonic acid is selected from

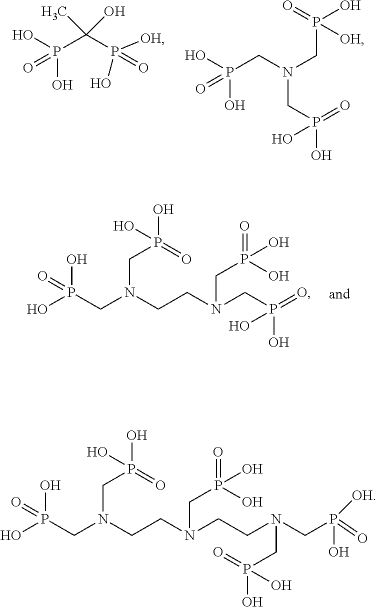 [SMILES: CC(O)(P(=O)(O)O)P(=O)(O)O ; O=P(O)(O)CN(CP(=O)(O)O)CP(=O)(O)O ; O=P(O)(O)CN(CCN(CP(=O)(O)O)CP(=O)(O)O)CP(=O)(O)O ; O=P(O)(O)CN(CCN(CP(=O)(O)O)CP(=O)(O)O)CCN(CP(=O)(O)O)CP(=O)(O)O]

Hydrocarbon based phosphates refer to cyclic (aromatic or non-aromatic) or acyclic linear or branched chain saturated or unsaturated compounds composed of a backbone having primarily hydrocarbon based units and one or more phosphate groups (i.e., three hydroxy moieties (P—OH) and one P═O double bond). For example, OPO(OH)₂ or PO₄³⁻ (either protonated or deprotonated). In one embodiment, hydrocarbon based phosphates are alkyl phosphates having the structural formula VII:

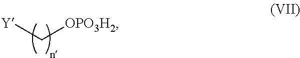

wherein Y′ is CH₃, OH, NH₂, SH, COOH, acrylate, or —OPO₃H₂; and n′ is an integer ranging from 8 to 20. In one embodiment, Y′ is CH₃ or OPO₃H₂. In one embodiment, n is an integer ranging from 12 to 18, wherein Y′ is as described for Formula VII. Alternatively, n′ is 12, 14, 16, or 18, wherein Y′ is as described for Formula VII. In one embodiment, an alkyl phosphate is selected from Lauryl phosphate (DPhate), Myristyl phosphate (TDPhate), Cetyl phosphate (HDPhate), and Stearyl phosphate (ODPhate).

Hydrocarbon based phosphate esters refer to cyclic (aromatic or non-aromatic) or acyclic linear or branched chain compounds composed of a backbone having primarily hydrocarbon based units and one or more phosphate ester groups i.e., a phosphorus atom bonded to four oxygen atoms (three single bonds and one double bond) with one of the oxygen atoms bound to another group e.g., RO—P(═O)(OH)₂.

In one embodiment, hydrocarbon based phosphonic acids (where X (C₁-C₄) alkylene) and hydrocarbon based phosphate esters (where X is O) are of the structural formula V:

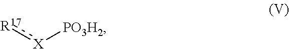

wherein X is O or a (C₁-C₄)alkylene; the dashed line is an optional double bond; R¹⁷ is selected from NR′R″ and (C₁-C₄)alkyl optionally substituted with 1 to 2 OH groups; and R′ and R″ are each independently selected from hydrogen and (C₁-C₄)alkyl optionally substituted with COOH. In one embodiment, X is O, CH₂, or CH, wherein the remaining variables are as described for Formula V. In one embodiment, R¹⁷ is NH₂, —NH(C₁-C₄) alkylCOOH, or (C₁-C₄)alkyl substituted with 2 OH groups, wherein the remaining variables are as described for Formula V. In one embodiment, X is O, CH₂, or CH; and R¹⁷ is NH₂, —NH(C₁-C₄)alkylCOOH, or (C₁-C₄)alkyl substituted with 2 OH groups, wherein the remaining variables are as described for Formula V. In one embodiment, an acyclic hydrocarbon based phosphonic acid is selected from

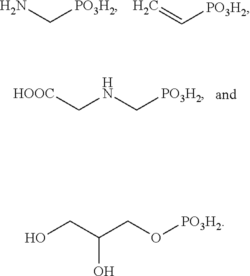

In one embodiment, an acyclic hydrocarbon based phosphonic acid is of the structural formula VI:

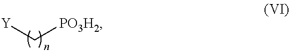

wherein Y is CH₃, OH, NH₂, SH, COOH, acrylate, or —PO₃H₂; and n is an integer ranging from 8 to 20. In one embodiment, Y is CH₃ or PO₃H₂. In one embodiment, n is an integer ranging from 12 to 18, wherein Y is as described for Formula VI. Alternatively, n is 12, 14, 16, or 18, wherein Y is as described for Formula VI. In one embodiment, an acyclic hydrocarbon based phosphonic acid is selected from CH₃(CH₂)₁₁PO₃H₂, CH₃(CH₂)₁₃PO₃H₂, CH₃(CH₂)₁₅PO₃H₂, CH₃(CH₂)₁₇PO₃H₂, and H₂PO₃ (CH₂)₁₂PO₃H₂.

Aromatic cyclic hydrocarbon based phosphate esters refer to aromatic rings comprised only of carbon atoms (e.g., phenyl) in which the ring is substituted by one or more phosphate groups. Additional substituents besides the one or more phosphate groups are permitted on the aryl ring. In one embodiment, an aromatic cyclic hydrocarbon based phosphate ester is

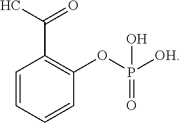

Polyphosphates refer to ionizable phosphorus containing compounds composed of phosphate structural units linked together by sharing oxygen atoms, including orthophosphorous acid in which the fourth oxygen atom is replaced by a hydrogen. In one embodiment, an acyclic polyphosphate is selected from

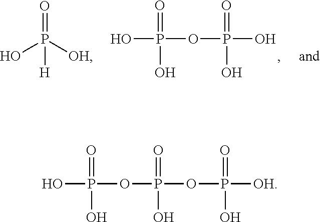

In one embodiment, a cyclic polyphosphate is selected from

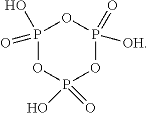 [SMILES: O=P1(O)OP(=O)(O)OP(=O)(O)O1]

As used herein, complex lipids refer to phosphorylated lipid compounds comprising more than two types of components, including an ionizable phosphate group and fatty acid, alcohol, or glycerol. Additional components may include carbohydrates and proteins. Complex lipids include, but are not limited to Myristyl malate phosphonic acid (MMPA), Phosphatidylcholine (PC), Phosphatidylinositol (Ptdlns), Cyclic phosphatidic acid (CYPA) and Linoleamidopropyl PG-Dimonium Chloride phosphate (LAP-PG DACP).

In one aspect, provided are methods for protecting hair from heat or chemical treatments (e.g., bleaching) comprising applying to the hair a hair care composition described herein.

In one aspect, the hair care composition is applied prior to, concurrently with, or after heat or chemical treatment (e.g., bleaching). In one aspect, the hair care composition is applied prior to heat or chemical treatment (e.g., bleaching).

As used herein, “strengthening” hair refers to an improvement in one or more thermal and/or mechanical properties of the hair resulting from treatment with an ionizable phosphorus containing compound described herein. Thermal and mechanical properties of the hair include, but are not limited to, denaturation temperature, Young's modulus, break extension, break stress, hair breakage, hair fracture patterns, and fiber fatigue.

As used herein, “repairing” hair refers to an improvement in hair structural integrity and/or surface quality. Such improvements include, but are not limited to, reduced swelling, reduced protein loss, increased surface hydrophobicity, enhanced surface smoothness, and improved humidity resistance.

As used herein, an “effective amount of an ionizable phosphorus containing compound” refers to an amount of an ionizable phosphorus containing compound that is sufficient to strengthen hair, repair, or protect hair as described herein. In one aspect, an effective amount of an ionizable phosphorus containing compound ranges from about 0.1 wt % to about 50 wt % based on the total weight of the hair care composition. In another aspect, an effective amount of an ionizable phosphorus containing compound ranges from about 0.1 wt % to about 20 wt % based on the total weight of the hair care composition. In another aspect, an effective amount of an ionizable phosphorus containing compound ranges from about 0.1 wt % to about 15 wt % based on the total weight of the hair care composition. In another aspect, an effective amount of an ionizable phosphorus containing compound ranges from about 2 wt % to about 50 wt % based on the total weight of the hair care composition. In another aspect, an effective amount of an ionizable phosphorus containing compound ranges from about 2 wt % to about 20 wt % based on the total weight of the hair care composition. In another aspect, an effective amount of an ionizable phosphorus containing compound ranges from about 2 wt % to about 15 wt % based on the total weight of the hair care composition. In another aspect, an effective amount of an ionizable phosphorus containing compound is about 2 wt %, about 5 wt %, or about 15 wt % based on the total weight of the hair care composition. In another aspect, an effective amount of an ionizable phosphorus containing compound is about 2 wt % based on the total weight of the hair care composition. In another aspect, an effective amount of an ionizable phosphorus containing compound ranges from about 0.2 wt % to about 5 wt % based on the total weight of the hair care composition. In another aspect, an effective amount of an ionizable phosphorus containing compound ranges from about 0.5 wt % to about 4 wt % based on the total weight of the hair care composition. In another aspect, an effective amount of an ionizable phosphorus containing compound ranges from about 0.5 wt % to about 3 wt % based on the total weight of the hair care composition. In another aspect, an effective amount of an ionizable phosphorus containing compound ranges from about 0.75 wt % to about 2 wt % based on the total weight of the hair care composition. In another aspect, an effective amount of an ionizable phosphorus containing compound ranges from about 0.75 wt % to about 1.25 wt % based on the total weight of the hair care composition. In another aspect, an effective amount of an ionizable phosphorus containing compound is about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2 wt %, based on the total weight of the hair care composition.

Hair care compositions described herein may comprise, in addition to an ionizable phosphorus containing compound, one or more cosmetically acceptable ingredients. Such ingredients include, but are not limited to, amino acids, amino acid derivatives, peptides, vitamins, keratins, acidifiers, polycarboxylic acids, fatty acids, fatty alcohols, fatty acid esters, emulsifiers, emollients, gelling agents, antioxidants, oils, waxes, preservatives, sunscreens, and polyphenols.

In additional aspects, an ionizable phosphorus containing compound may be applied as part of an aqueous solution (e.g., without additional cosmetically acceptable ingredients) or a hair care composition. In one aspect, an effective amount of an ionizable phosphorus containing compound comprises the ionizable phosphorus containing compound as part of an aqueous solution. In one aspect, an effective amount of an ionizable phosphorus containing compound comprises applying at least about a 0.1 wt % ionizable phosphorus containing compound solution or hair care composition to the hair (e.g., such as a 1.5 g hair tress). In another aspect, an effective amount of an ionizable phosphorus containing compound comprises applying about 0.1 wt % to about 50 wt % ionizable phosphorus containing compound solution or hair care composition to the hair (e.g., such as a 1.5 g hair tress). In another aspect, an effective amount of an ionizable phosphorus containing compound comprises applying about 0.5 wt % to about 50 wt % ionizable phosphorus containing compound solution to the hair (e.g., such as a 1.5 g hair tress). In another aspect, an effective amount of an ionizable phosphorus containing compound comprises applying at least about a 2 wt % aqueous ionizable phosphorus containing compound solution to the hair (e.g., such as a 1.5 g hair tress). In another aspect, an effective amount of an ionizable phosphorus containing compound comprises applying about 2 wt % to about 50 wt % aqueous ionizable phosphorus containing compound solution to the hair (e.g., such as a 1.5 g hair tress). In another aspect, an effective amount of an ionizable phosphorus containing compound comprises applying about 2 wt % to about 20 wt % aqueous ionizable phosphorus containing compound solution to the hair (e.g., such as a 1.5 g hair tress). In another aspect, an effective amount of an ionizable phosphorus containing compound comprises applying about 2 wt % to about 15 wt % aqueous ionizable phosphorus containing compound solution to the hair (e.g., such as a 1.5 g hair tress). In another aspect, an effective amount of an ionizable phosphorus containing compound comprises applying about 2 wt % to about 10 wt % aqueous ionizable phosphorus containing compound solution to the hair (e.g., such as a 1.5 g hair tress). In another aspect, an effective amount of an ionizable phosphorus containing compound comprises applying about 0.5 wt % to about 1 wt % aqueous ionizable phosphorus containing compound solution to the hair (e.g., such as a 1.5 g hair tress). In another aspect, an effective amount of an ionizable phosphorus containing compound comprises applying about 1 wt % to about 3 wt % aqueous ionizable phosphorus containing compound solution to the hair (e.g., such as a 1.5 g hair tress). In another aspect, an effective amount of an ionizable phosphorus containing compound comprises applying about 2 wt %, about 5 wt %, about 10 wt %, about 15 wt %, or about 20 wt % aqueous ionizable phosphorus containing compound solution to the hair (e.g., such as a 1.5 g hair tress). In another aspect, an effective amount of an ionizable phosphorus containing compound comprises applying about 0.5 wt %, 2 wt %, about 5 wt %, about 10 wt %, about 15 wt %, or about 20 wt % aqueous ionizable phosphorus containing compound solution to the hair (e.g., such as a 1.5 g hair tress). In another aspect, an effective amount of an ionizable phosphorus containing compound comprises applying about 0.75 wt % or about 2 wt %, aqueous ionizable phosphorus containing compound solution to the hair (e.g., such as a 1.5 g hair tress). In another aspect, an effective amount of an ionizable phosphorus containing compound comprises applying about 2 wt % aqueous ionizable phosphorus containing compound solution to the hair (e.g., such as a 1.5 g hair tress). In another aspect, an effective amount of an ionizable phosphorus containing compound comprises applying about 0.75 wt % aqueous ionizable phosphorus containing compound solution to the hair (e.g., such as a 1.5 g hair tress).

When a range is described, it is to be understood that all ranges and individual data points, including the end points, are intended. For example, about 1 wt % to about 3 wt % covers about 1 wt % to about 2 wt %, about 1.5 wt % to about 2 wt %, about 2 wt % to about 3, about 2.5 wt % to about 3 wt %, about 1 wt %, about 1.1 wt %, about 1.2 wt %, about 1.3 wt %, about 1.4 wt %, about 1.5 wt %, . . . about 2 wt %, and all other ranges and data points in between.

In one aspect, the pH of the hair care composition or aqueous solution comprising an ionizable phosphorus containing compound can be adjusted depending on the need. For example, in certain aspects the pH of the hair care composition or aqueous solution comprising an ionizable phosphorus containing compound ranges from about pH 2 to about pH 10 e.g., from about pH 2 to about pH 5, from about pH 3 to about pH 5, and from about pH 4 to about pH 5. In certain aspects the pH of the hair care composition or aqueous solution comprising an ionizable phosphorus containing compound is about pH 2, about pH 3, about pH 4, about pH 4.5, or about pH 5.

As discussed above, and shown in the exemplification section below, applying an ionizable phosphorus containing compound to the hair results in improvements in the thermal properties of the hair e.g., an increase in denaturation temperature and/or better humidity resistance. In some aspects, the increase in denaturation achieved following the application of an ionizable phosphorus containing compound to the hair is 3° C. or more, 4° C. or more, or 5° C. or more.

Also, as discussed above, and shown in the exemplification section below, applying an ionizable phosphorus containing compound to the hair results in improvements in the mechanical properties of the hair e.g., an increase in break stress and/or Young's modulus. In some aspects, the increase in break stress achieved following the application of an ionizable phosphorus containing compound to the hair is 3% or greater, 4% or greater, 5% or greater, 6% or greater, 7% or greater, 8% or greater, or 9% or greater. In some aspects, the increase in break stress achieved following the application of an ionizable phosphorus containing compound to the hair ranges from 3% to about 30% or from about 5% to about 30%. In some aspects, the increase in Young's modulus achieved following the application of an ionizable phosphorus containing compound to the hair is 3% or greater, 4% or greater, 5% or greater, 6% or greater, 7% or greater, 8% or greater, or 9% or greater. In some aspects, the increase in Young's modulus achieved following the application of an ionizable phosphorus containing compound to the hair ranges from 3% to about 30% or from about 5% to about 30%.

Also, as discussed above, and shown in the exemplification section below, applying an ionizable phosphorus containing compound to the hair results in an increase in the surface hydrophobicity of the hair. In some aspects, the increase in surface hydrophobicity achieved following the application of an ionizable phosphorus containing compound to the hair is defined by an increase in the water contact angle as measured by a drop shape analyzer (DSA 100M). Also, as discussed above, and shown in the exemplification section below, applying an ionizable phosphorus containing compound to the hair results in an increase in hair surface smoothness. In some aspects, the increase in surface smoothness achieved following the application of an ionizable phosphorus containing compound to the hair is defined by a reduced coefficient of friction (COF) as measured by Dia-Stron Fibra.one.

### EXEMPLIFICATION

The following examples are provided by way of illustration and not limitation.

A. Ionizable Phosphorus Containing Molecules with Multi-Phosphonic Acid Groups

To demonstrate internal strengthening and repair benefits of phosphate-based treatments, a series of ionizable phosphorus containing molecules with multi-phosphonic acid groups were screened. The molecules tested herein include 1-hydroxyethylidene-(1,1-diphosphonic acid) (HEDP), Nitrilotrimethyl-phosphonic acid (NTMP), Ethylenediaminetetra-(methylene phosphonic acid) (EDTMP), and Diethylenetriaminepenta-(methylene phosphonic acid) (DTPMP), which contains 2, 3, 4, and 5 phosphonic acid groups, respectively.

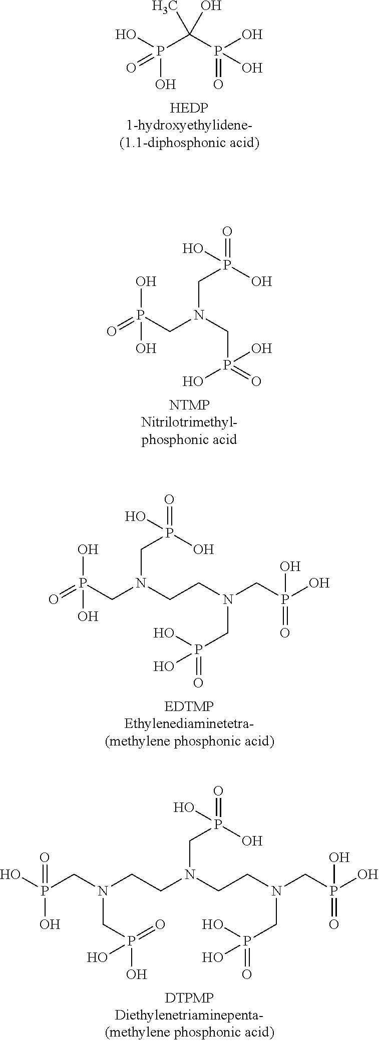 [SMILES: CC(O)(P(=O)(O)O)P(=O)(O)O ; O=P(O)(O)CN(CP(=O)(O)O)CP(=O)(O)O ; O=P(O)(O)CN(CCN(CP(=O)(O)O)CP(=O)(O)O)CP(=O)(O)O ; O=P(O)(O)CN(CCN(CP(=O)(O)O)CP(=O)(O)O)CCN(CP(=O)(O)O)CP(=O)(O)O]

European virgin brown hair was bleached three times to obtain the 3× bleached hair model. Each bleaching was done with a mixture of bleaching powder (BW2) and 40V clear developer at 1:2 ratio (5 g mixture per 1.5 g of hair) for 1 hour at room temperature, followed by 30 s water rinse, 1× wash with Sodium Lauryl Ether Sulfate (SLES), and dried in a humidity chamber for 15 min at 50° C., 20% RH. The 3× bleached hair was then further treated with a 2 wt % phosphonic acid aqueous solution at pH of 2 for 10 min, followed by blow dry (10 passes with a round brush using a Dyson hair drier at high heat, medium fan speed). Hair denaturation temperature (Ta) before and after the treatment was measured using a differential scanning calorimeter (DSC) to demonstrate hair internal repair and strengthening benefits provided by these phosphonic acid molecules. For each DSC measurement, a 3-5 mg of hair sample in 50 μL Deionized water is sealed and equilibrated (overnight) in a stainless-steel pressure resistant high-volume pan, then heated from 25° C. to 180° C. at a 5° C./min heating rate.

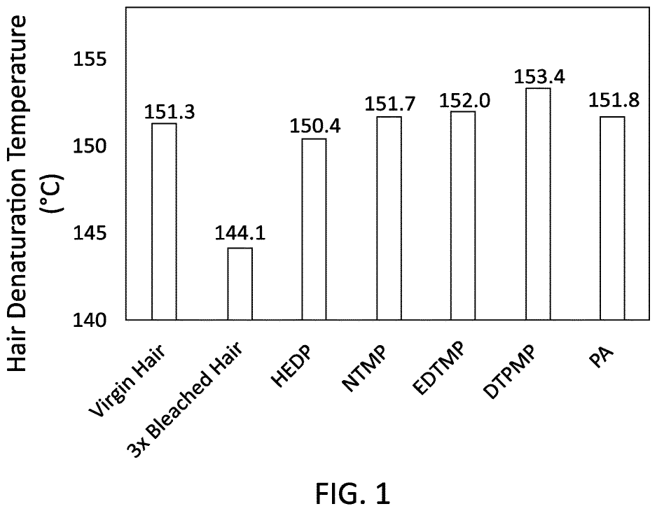

As shown in FIG. 1, compared to virgin hair, the 3× bleached hair showed a great decrease in Ta due to severe damage caused by bleaching process. However, once the bleached hair is further treated with the multi-phosphonic acid molecules, Tais fully recovered to or beyond the virgin level, suggesting the strong internal structural repair and strengthening benefits provided by these molecules.

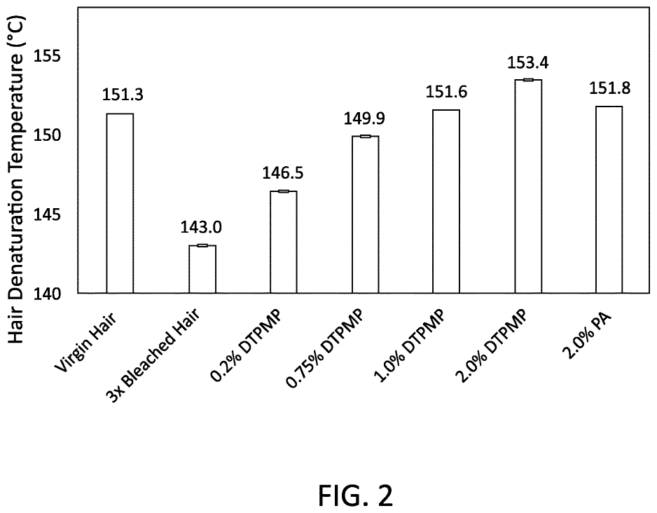

Hair treated with DTPMP, the molecule with 5 phosphonic acid groups, showed the highest Ta (153.4° C.), even higher than that was previously reported by phytic acid. Hence, it was further explored at different concentrations varying from 0.2 to 2 wt % to demonstrate its efficacy. FIG. 2 shows a clear positive dose response relationship between the DTPMP concentration and T_{d}. Tresses treated with DTPMP with concentrations above 0.75 wt % all showed T_{d} similar to or beyond the virgin level, indicating effective internal repair and complete restore by DTPMP.

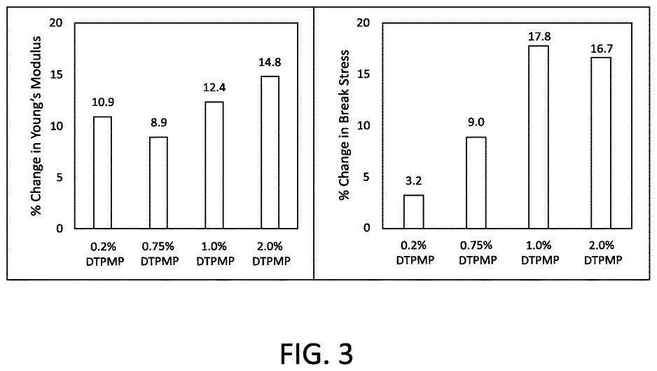

Additionally, the strengthening benefits of DTPMP were also evaluated by Dia-Stron single fiber mechanical testing. All single fiber tensile measurements are performed using a Dia-Stron MTT686/MTT690 (under 100% extension at gauge force of 2N, break threshold of 5 g, rate of 20 mm/min, maximum force of 2000 gmf and premature failure threshold of 10% strain). Before the tensile testing, all hair fibers (30 mm in length, ≥20 fibers per treatment) are fixed by 2 brass crimps at the ends and mounted on a Dia-Stron tray. Initial dimension measurements are collected using a Dia-Stron FDAS770 (double ended samples, sample size of 30 mm in length, no of slices of 5 under one rotation per slice), followed by equilibration in Deionized water for at least 2 h prior to testing. FIG. 3 shows that both Young's Modulus and break stress were significantly improved when bleached hair was treated with DTPMP solutions at concentrations of 0.2 to 2.0 wt % followed by blow dry. The maximum strengthening was achieved when DTPMP is used at 1.0-2.0 wt %.

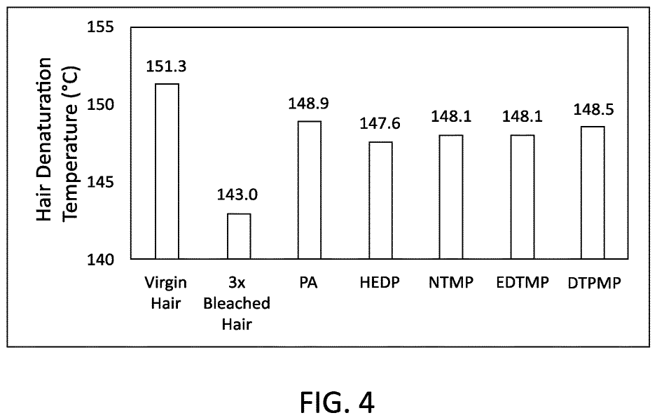

In another study, phosphonic acid treatments were performed at a higher pH of 4.0-4.5 for 10 min followed by blow dry. Similar to what were observed at pH 2, all treated hair samples showed higher T_{d} compared to the bleached hair (FIG. 4).

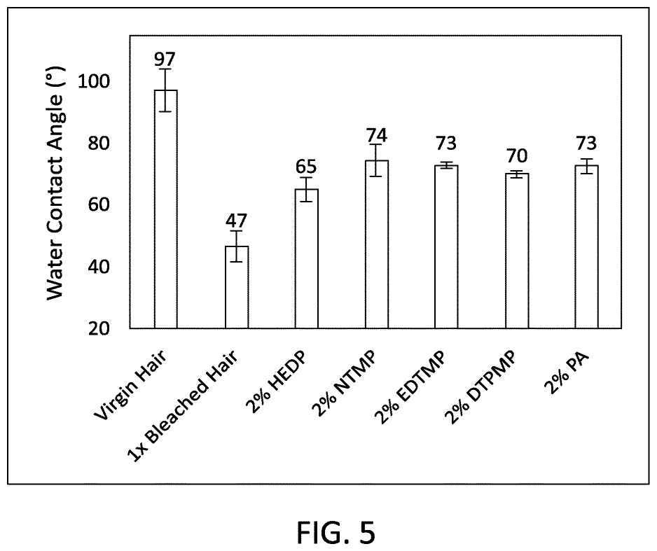

To demonstrate the surface repairing capabilities of these multi-phosphonic acid molecules, the DSA100M (Kruss Scientific) Drop Shape Analyzer was used to evaluate hair surface hydrophobicity change before and after treatments by measuring water contact angles. For this test, all the treatments were performed on 1× bleached hair, which was prepared by treating European virgin brown hair with a mixture of BW2 bleaching powder and clear 30 V developer (1:1.5 ratio, 5 g mixture per 1.5 g of hair) for 20 min under a Belvedere hooded dryer with high heat setting, followed by 30 s water rinse, 30 s 10% SLES wash, and air dry at room temperature. The 1× bleached hair was treated with 2 wt % active solution at pH 4-4.5, kept for 10 min, followed by air dry until 100% dry. In the DSA method, a single hair fiber is mounted in a sample holder and focused by the digital camera of DSA100M. Then a droplet of filtered DI water (20-60 nL) is applied to the top of the hair fiber by a digital droplet applier, A video is then recorded until the disappearance of the water droplet. The water contact angle is obtained using the first image of the water droplet recorded. The data is collected for 3 fibers per treatment and 3 points per fiber, a total of 9 data points per treatment. A low water contact angle was observed for the 1× bleached hair, which corresponds to a ~52% reduction in hair surface hydrophobicity compared to virgin hair (FIG. 5). However, much higher contact angles were achieved after the multi-phosphonic acid treatments, indicating effective enhancements in hair surface hydrophobicity by these molecules.

B. Ionizable Phosphorus Containing Molecules with at Least One Other Functional Group

Another series of ionizable phosphorus containing molecules with at least one other functional group such as amine, carboxyl, hydroxyl, and vinyl groups was explored. The five molecules tested were Aminomethyl phosphonic acid (AMPA), N-(phosphonomehyl)glycine (PMG), Glycerol-3-phosphate (G3P), Vinyl phosphonic acid (VPA) and 2-(phosphonoxy)benzoic acid (2PPBA).

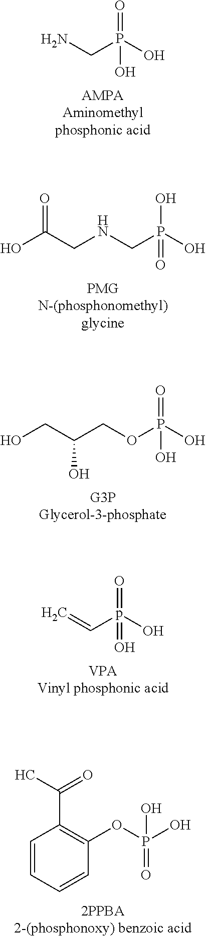

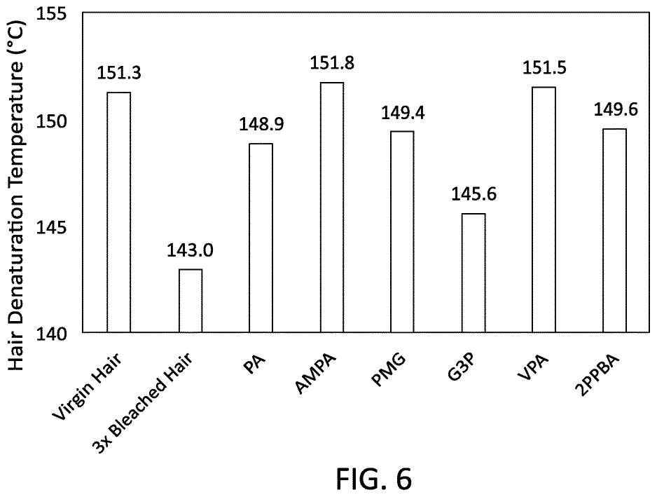

All treatments were performed on 3× bleached hair with a 2 wt % active aqueous solution at pH 4.0-4.5 for 10 min followed by blow dry. As shown in FIG. 6, compared to virgin hair, the 3× bleached hair showed a great decrease in T_{d} due to severe damage caused by bleaching process. However, once the bleached hair is further treated with a 2 wt % active solution, both AMPA and VPA treatments showed a full recovery of T_{d} to the virgin level, suggesting the strong structural repair and strengthening benefits of these molecules, superior to phytic acid (PA). Additionally, PMG and 2PPBA also showed a similar strengthening benefit as PA.

### C. Polyphosphates

Another group of molecules explored were polyphosphates, in particular Orthophosphorous acid (OPA), Sodium pyrophosphate tetrabasic acid (TSPP), Sodium triphosphate pentabasic acid (STPPB), and Trisodium trimetaphosphate (STMP).

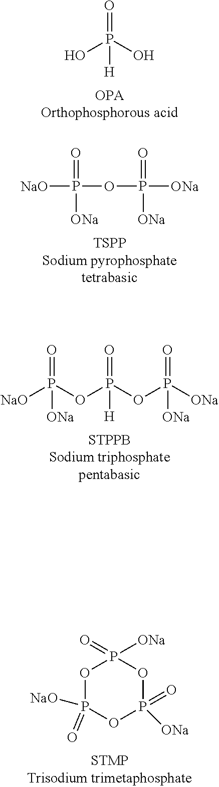

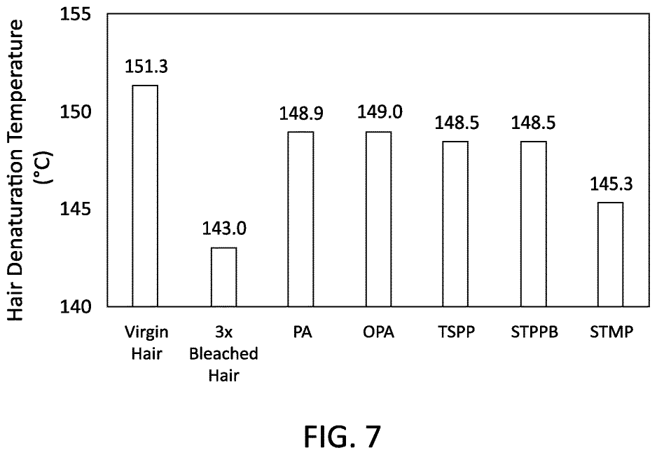

All treatments were performed on 3× bleached hair with a 2 wt % active aqueous solution at pH 4.0-4.5 for 10 min followed by blow dry. As shown in FIG. 7, 3x bleached hair treated with the polyphosphate solutions all showed great improvements in T_{d}. Except for STMP, all polyphosphates showed similar strengthening benefits as phytic acid (PA).

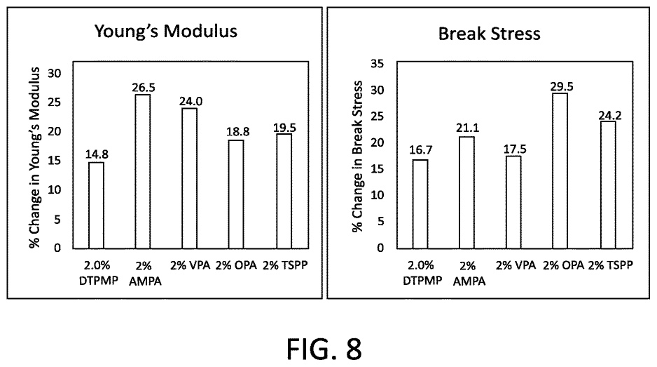

Mechanical properties of hair fibers treated with selected lead ionizable phosphorus containing molecules including a multi-phosphonic acid molecule (DTPMP), molecules with at least one other functional group (AMPA and VPA) and polyphosphate molecules (OPA and TSPP), are shown in FIG. 8. Overall, all the molecules tested showed significant increases in both Young's modulus and break stress. Phosphonic acids like VPA and AMPA, with low molecular weight and an additional functional group with the ability to form strong covalent bonds with hair functional groups led to the biggest increases in Young's modulus, similar to the DSC results.

### D. Alkyl Phosphonic Acids

Alkyl phosphonic acids are organic molecules that share a common phosphate core structure and vary by the identity of the alkyl chains (ranging from 8 to 22 carbons in length) connected via a carbon-phosphorus bond. Mono alkyl phosphonic acids with alkyl chains ranging from 12 to 18 carbons in length and a bis alkyl phosphonic acid were explored. The molecules tested were n-dodecylphosphonic acid (DPA, C-12), Tetradecyl phosphonic acid (TDPA, C-14), Hexadecyl phosphonic acid (HDPA, C-16), Octadecyl phosphonic acid (ODPA, C-18), and (12-phosphonododecyl) phosphonic acid (PDDPA, C-12).

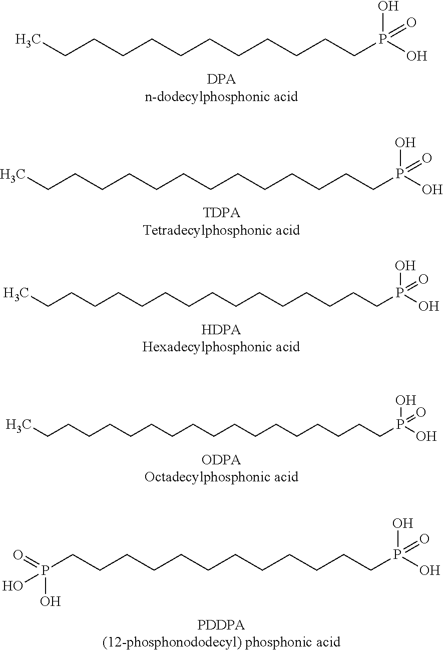 [SMILES: CCCCCCCCCCCCP(=O)(O)O ; CCCCCCCCCCCCCCP(=O)(O)O ; CCCCCCCCCCCCCCCCP(=O)(O)O ; CCCCCCCCCCCCCCCCCCP(=O)(O)O ; O=P(O)(O)CCCCCCCCCCCCP(=O)(O)O]

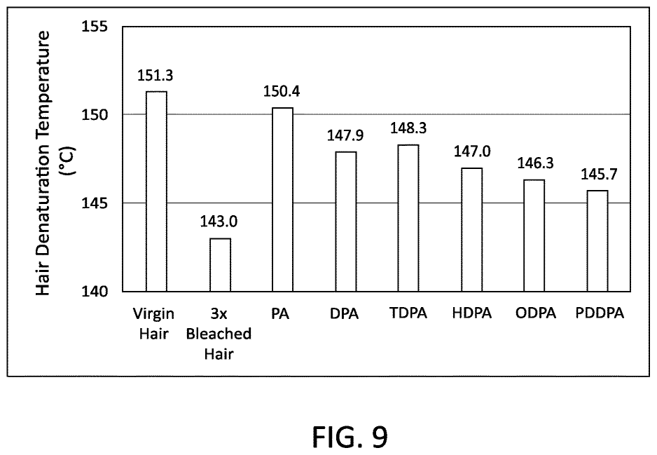

To demonstrate the strengthening benefits of alkyl phosphonic acids, DSC method is used to measure the hair denaturation temperature. All treatments were performed on 3× bleached hair treated with an ethanolic alkyl phosphonic acid solution containing 2 wt % DPA, TDPA, or HDPA, or an ethanol-chloroform mixture containing 2 wt % ODPA for 10 min followed by blow dry. As shown in FIG. 9, 3× bleached hair treated with the alkyl phosphonic acid solutions all showed great improvements in T_{d}. A trend of decrease in T_{d} with increasing alkyl chain from carbon 12 to 18 is also observed, likely due to less effective penetration for the longer alkyl chain phosphonic acids.

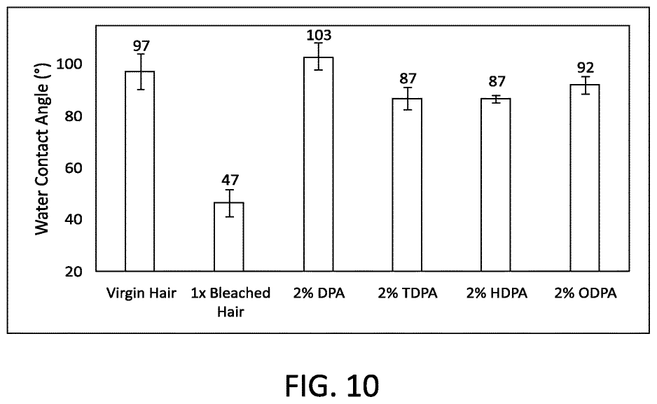

To demonstrate the surface repairing capabilities of these alkyl phosphonic acids, the DSA100M (Kruss Scientific) Drop Shape Analyzer was used to evaluate hair surface hydrophobicity change before and after treatments by measuring water contact angles. All treatments were performed on 1× bleached hair. The 1× bleached hair was treated with an ethanol solution containing 2 wt % DPA, TDPA, HDPA, or an ethanol-chloroform mixture containing 2 wt % ODPA, kept for 10 min, followed by air dry until 100% dry. A low water contact angle was observed for the 1× bleached hair, which corresponds to a ~52% reduction in hair surface hydrophobicity compared to virgin hair (FIG. 10). However, the contact angle was recovered back to or even beyond the virgin level after each alkyl phosphonic acid treatment, indicating an effective recovery of hair surface hydrophobicity.

Furthermore, to demonstrate the hair surface smoothness enhancement by these alkyl phosphonic acids, Dia-Stron Fibra.one was used to evaluate hair surface friction change before and after treatments by measuring coefficient of friction (COF). All treatments were performed on 18-MEA (18-Methyl Eicosanoic Acid) removed hair, which was prepared by treating European virgin brown hair with 1M methanolic KOH for 30 min followed with 10% SLES wash and air dry until 100% dry. The 18-MEA removed hair was further treated with an ethanol solution containing 2 wt % DPA, TDPA, or HDPA, or an ethanol-chloroform mixture containing 2 wt % ODPA, and kept for 10 min followed by air dry until 100% dry.

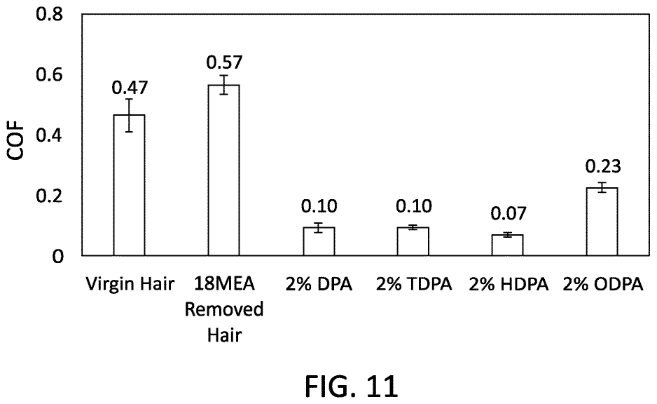

As shown in FIG. 11, hair surface becomes rougher/coarser when 18-MEA is removed from hair surface as indicated by the higher COF compared to virgin hair. However, COF was reduced drastically and even much lower than virgin level after the alkyl phosphonic acid treatment, indicating a significant improvement in hair surface smoothness. Together with the surface hydrophobicity measurement data, these results suggest that these alkyl phosphonic acids can provide superior surface repair benefits.

### E. Alkyl Phosphates

Alkyl phosphates are organic molecules containing a phosphate group attached to an alkyl chain via an oxygen-phosphorous bond, where the alkyl chain can vary (e.g., from 8 to 20 carbons in length). Alkyl phosphates with alkyl chains ranging from 12 to 18 carbons in length including Lauryl phosphate (DPhate), Myristyl phosphate (TDPhate), Cetyl phosphate (HDPhate), Stearyl phosphate (ODPhate) were explored.

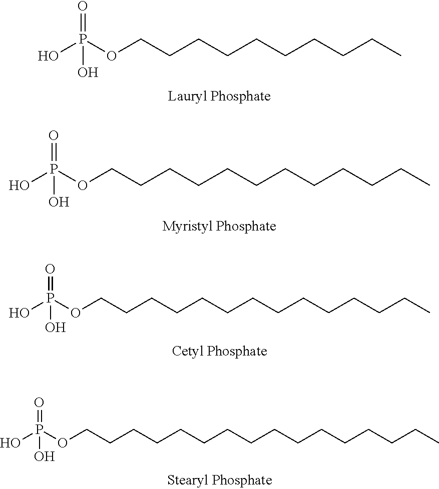 [SMILES: CCCCCCCCCCOP(=O)(O)O ; CCCCCCCCCCCCOP(=O)(O)O ; CCCCCCCCCCCCCCOP(=O)(O)O ; CCCCCCCCCCCCCCCCOP(=O)(O)O]

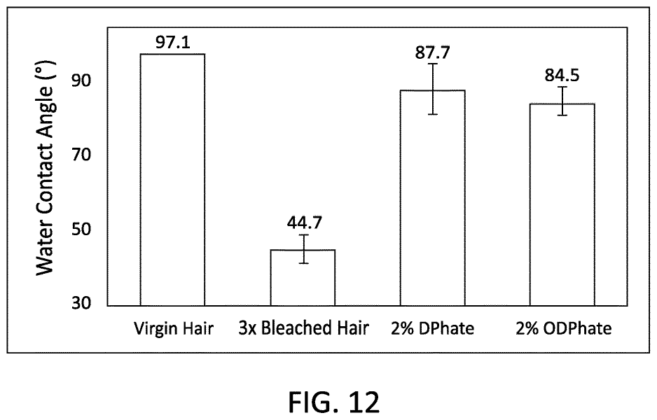

To demonstrate hair surface repair benefits by these alkyl phosphates, water contact angle measurements were performed on virgin hair, 3× bleached hair, and 3× bleached hair treated with an ethanol solution containing 2 wt % of DPhate and ODPhate, respectively, for 10 min, followed by blow dry. A very low water contact angle was observed for the 3× bleached hair, which corresponds to about 54% reduction in hair surface hydrophobicity compared to virgin hair (FIG. 12). However, once the 3× bleached hair was further treated with DPhate or ODPhate, the contact angle was recovered significantly and close to the virgin level, indicating an effective recovery of hair surface hydrophobicity by these alkyl phosphates.

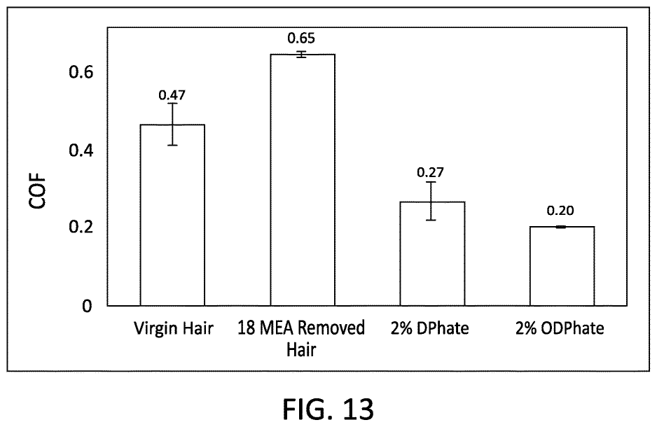

Surface friction measurement (FIG. 13) shows that the coefficient of friction (COF) of the 18-MEA removed was reduced drastically upon the alkyl phosphate treatment, even lower than virgin level, indicating a significant improvement in hair surface smoothness. Together with the surface hydrophobicity measurement data, these results suggest that these alkyl phosphates can provide superior surface repair and smoothing benefits.

### F. Other Phosphorus Containing Compounds

Other phosphorus containing compounds such as phosphorylated complex lipids were also explored. In this study, complex lipids such as Myristyl malate phosphonic acid (MMPA), Phosphatidylcholine (PC), Phosphatidylinositol (Ptdlns), Cyclic phosphatidic acid (CYPA) and Linoleamidopropyl PG-Dimonium Chloride phosphate (LAP-PG DACP) were tested.

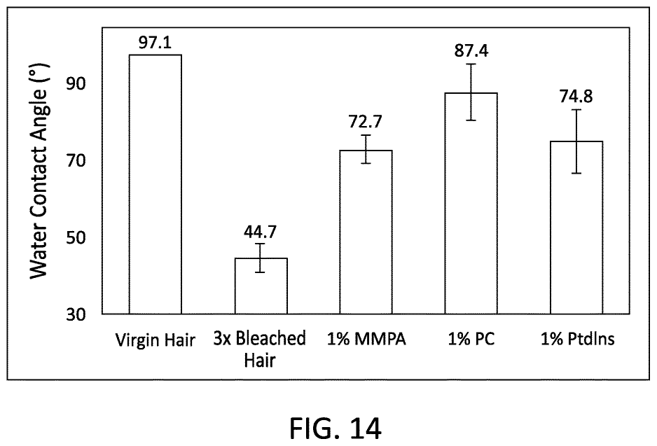

In this study, 3× bleached hair was treated with an aqueous solution containing 1 wt % active. As shown in the FIG. 14, complex lipids like MMPA, PC, and Ptdlns increased the water contact angle by at least 60%, more particularly for the PC-treated hair, the contact angle was recovered close to the virgin level, indicating the most recovery of hair surface hydrophobicity by PC.

### G. A Combination for Both Internal and Surface Repair

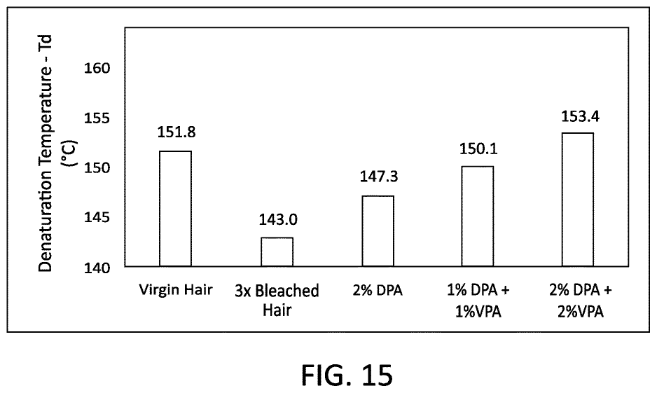

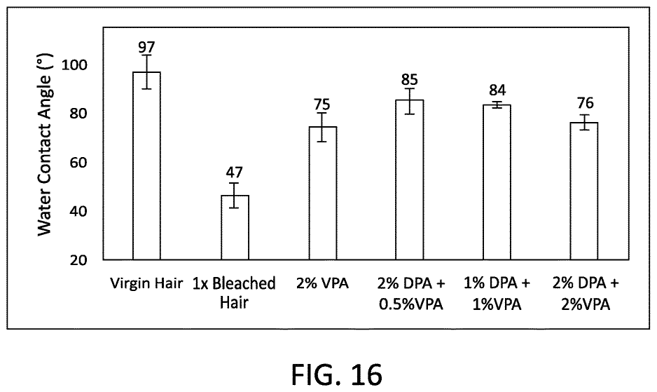

A combination of two or more ionizable phosphorus containing molecules to achieve maximal internal strengthening and surface repair was explored. As an example, a mixture of vinyl phosphonic acid (VPA) as an effective internal cross-linker and n-dodecyl phosphonic acid (DPA) as a strong surface repair agent was tested. The treatments were performed on 3× bleached hair with an ethanol solution containing 1-2 wt % DPA and 0.5-2 wt % VPA for 10 min followed by blow dry. As shown in FIG. 15, the addition of VPA into DPA effectively boosted hair T_{d} compared to the DPA treatment alone. On the other hand, as shown in FIG. 16, the addition of DPA into VPA could further enhance hair surface hydrophobicity. At the preferred concentration of 1 wt % DPA and 1 wt % VPA, both high T_{d} and contact angle were achieved, suggesting maximal internal structural strengthening and external surface repair provided by the mixture.

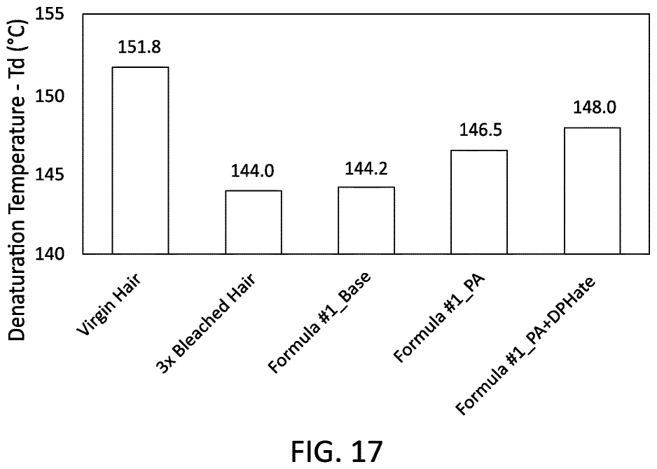

A combination of phosphorus containing compounds was also explored in hair care formulations. In this experiment, two different formulation prototypes, Formula #1 and Formula #2, were prepared by incorporating phytic acid (PA) at 0.75 wt % with or without DPhate at 1 wt %. To demonstrate the strengthening benefits of treatment with a combination of two or more phosphorus containing molecules, DSC method was used to measure the hair denaturation temperature (T_{d}). All treatments were performed on 3× bleached hair and in each case the 3× bleached hair was first washed with LP basic shampoo and conditioner, followed by the treatment with a 0.25 g of formula for 10 min and blow dried with a round brush at high heat medium fan setting. As shown in FIG. 17, the incorporation of PA into the formula significantly increased T_{d} compared to the base formula, especially when combined with DPhate. This suggests enhanced strengthening benefits from using both phosphorus-containing molecules.

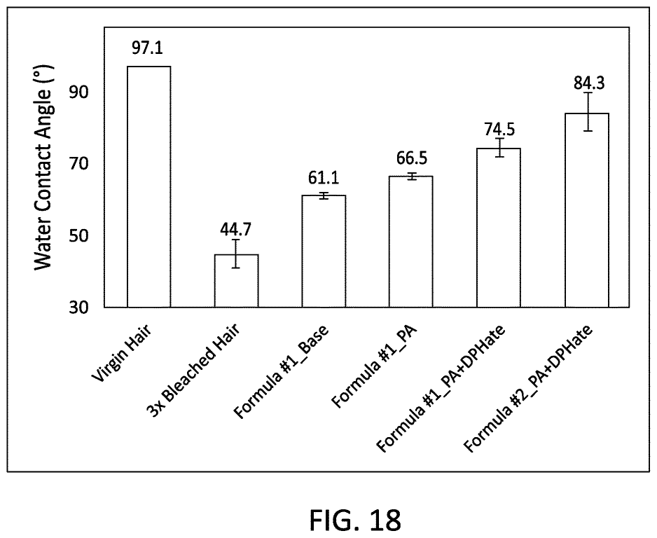

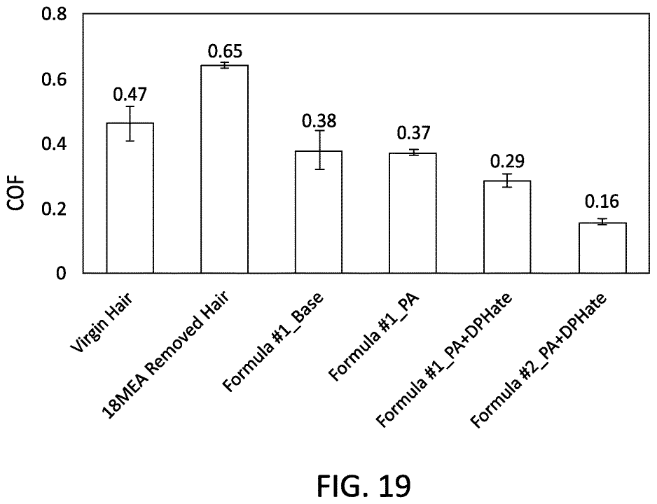

To demonstrate the surface repair benefits, water contact angle measurement was also performed. FIG. 18 shows that the addition of PA into the formula significantly improved hair surface hydrophobicity, especially when combined with DPhate. Similarly, surface friction measurement (FIG. 19) also shows that COF of the 18-MEA removed hair was reduced most effectively when treated with formulas containing both PA and DPhate. Together with the DSC measurement data, these results suggest that the combination of multiple phosphorus containing molecules can provide maximal cortex to cuticle hair repair. While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A hair care composition comprising an effective amount of an ionizable phosphorus containing compound selected from a hydrocarbon based phosphonic acid, a hydrocarbon based phosphate, a hydrocarbon based phosphate ester, a complex lipid, or a polyphosphate, or a combination of any of the foregoing.

2. The hair care composition of claim 1, wherein the ionizable phosphorus containing compound is an acyclic hydrocarbon based phosphonic acid, an acyclic hydrocarbon based phosphate, or an acyclic hydrocarbon based phosphate ester.

3. The hair care composition of claim 1, wherein the ionizable phosphorus containing compound comprises at least two phosphonic acid groups, at least three phosphonic acid groups, at least four phosphonic acid groups, or at least five phosphonic acid groups.

4-5. (canceled)

6. The hair care composition of claim 1, wherein the ionizable phosphorus containing compound comprises one or more nitrogen atoms.

7-11. (canceled)

12. The hair care composition of claim 1, wherein the ionizable phosphorus containing compound is of the structural formula I, II, III, or IV: 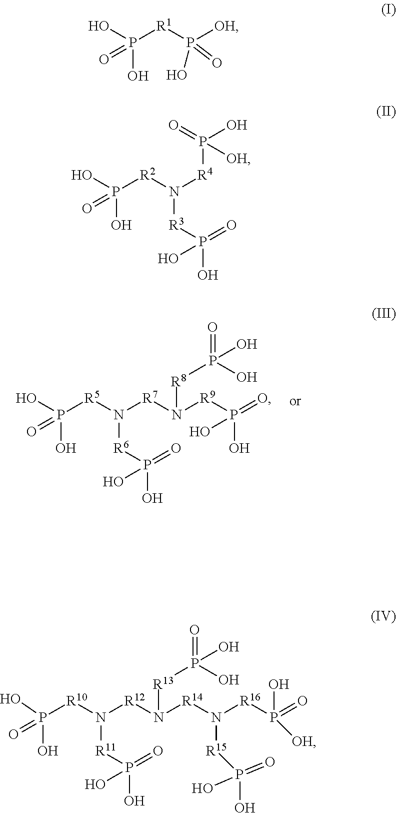
wherein each of R¹ to R⁸ are independently selected from an optionally substituted (C₁-C₆) alkylene.

13. (canceled)

14. The hair care composition of claim 12, wherein each of R¹ to R¹⁶ are independently selected from a (C₁-C₃)alkylene optionally substituted with OH.

15-16. (canceled)

17. The hair care composition of claim 1, wherein the ionizable phosphorus containing compound is selected from

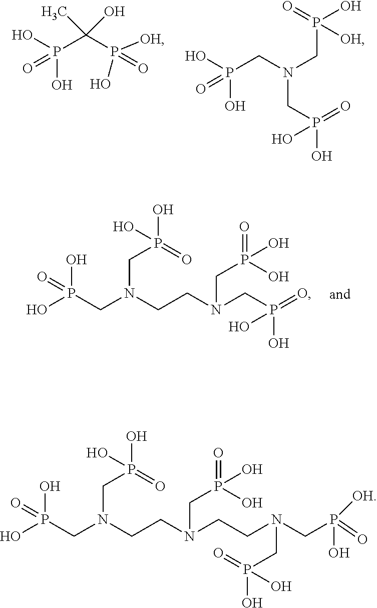 [SMILES: CC(O)(P(=O)(O)O)P(=O)(O)O ; O=P(O)(O)CN(CP(=O)(O)O)CP(=O)(O)O ; O=P(O)(O)CN(CCN(CP(=O)(O)O)CP(=O)(O)O)CP(=O)(O)O ; O=P(O)(O)CN(CCN(CP(=O)(O)O)CP(=O)(O)O)CCN(CP(=O)(O)O)CP(=O)(O)O]

18. The hair care composition of claim 1, wherein the ionizable phosphorus containing compound is of the structural formula V: 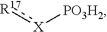
wherein
X is O or a (C₁-C₄)alkylene;
the dashed line is an optional double bond;
R¹⁷ is selected from NR′R″ and (C₁-C₄)alkyl optionally substituted with 1 to 2 OH groups; and
R′ and R″ are each independently selected from hydrogen and (C₁-C₄)alkyl optionally substituted with COOH.

19-20. (canceled)

21. The hair care composition of claim 1, wherein the ionizable phosphorus containing compound is selected from

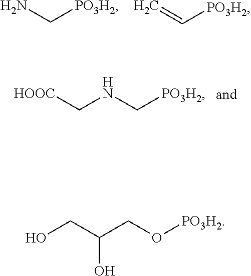

22. The hair care composition of claim 1, wherein the ionizable phosphorus containing compound is of the structural formula VI: 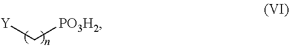
wherein Y is CH₃ or —PO₃H₂; and n is an integer ranging from 8 to 20.

23-24. (canceled)

25. The hair care composition of claim 1, wherein the ionizable phosphorus containing compound is selected from CH₃(CH₂)₁₁PO₃H₂, CH₃(CH₂)₁₃PO₃H₂, CH₃(CH₂)₁₅PO₃H₂, CH₃(CH₂)₁₇PO₃H₂, and H₂PO₃ (CH₂)₁₂PO₃H₂.

26-27. (canceled)

28. The hair care composition of claim 1, wherein the ionizable phosphorus containing compound is

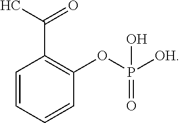

29. (canceled)

30. The hair care composition of claim 1, wherein the ionizable phosphorus containing compound is selected from

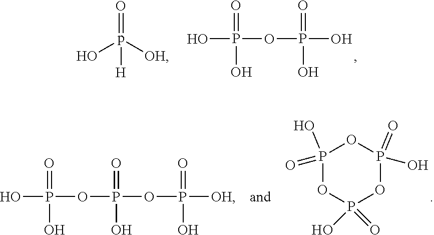

31. The hair care composition of claim 1, wherein the ionizable phosphorus containing compound is of the structural formula VII: 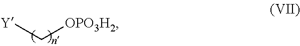
wherein Y′ is CH₃ or —OPO₃H₂; and n′ is an integer ranging from 8 to 20.

32-36. (canceled)

37. The hair care composition of claim 1, wherein the effective amount the ionizable phosphorus containing compound ranges from about 0.1 wt % to about 50 wt % based on the total weight of the hair care composition.

38-47. (canceled)

48. A method of strengthening or repairing hair comprising applying to the hair a hair care composition of claim 1.

49. A method of protecting hair from heat or chemical treatments comprising applying to the hair a hair care composition of claim 1.

50-51. (canceled)

52. The method or use of claim 48, wherein the hair is human hair.

53. The method or use of claim 48, wherein:
(i) the hair has been bleached prior to applying the hair care composition; or
(ii) the hair has been reduced prior to applying ionizable phosphorus containing compound; or
(iii) the hair has been color treated prior to applying the ionizable phosphorus containing compound.

54-55. (canceled)
